(19) United States
(12) Patent Application Publication
(10) Pub. No.: US 2014/0039312 A1
(43) Pub. Date: Feb. 6, 2014

(54) PACING-SITE SELECTION FOR LEAD PLACEMENT

(71) Applicant: Cardiac Pacemakers, Inc., St. Paul, MN (US)

(72) Inventors: Holly E. Rockweiller, Minneapolis, MN (US); Rodney W. Salo, Fridley, MN (US); Bruce A. Tockman, Scandia, MN (US); Lewis J. Thomas, III, Palo Alto, CA (US); Aaron R. McCabe, Edina, MN (US); Brian D. Soltis, St. Paul, MN (US); Darrell L. Rankin, Milpitas, CA (US); Michael S. Arney, Minneapolis, MN (US); Alex J. Sepulveda, Guaynabo, PR (US)

(73) Assignee: Cardiac Pacemakers, Inc., St. Paul, MN (US)

(21) Appl. No.: 13/953,904

(22) Filed: Jul. 30, 2013

Related U.S. Application Data
(60) Provisional application No. 61/736,985, filed on Dec. 13, 2012.
(60) Provisional application No. 61/678,693, filed on Aug. 2, 2012.

(51) Int. Cl. A61N 1/372

(52) U.S. Cl. CPC A61N 1/372
USPC 600/437; 600/508; 600/509; 600/479; 600/485; 607/17

(57) ABSTRACT

Techniques are disclosed for pacing site selection. In one example, a method includes using a sensing element such as an ultrasonic transducer, an optical pressure sensor, a MEMS pressure sensor, a SAW pressure sensor, an accelerometer, a gyroscope, or any other suitable sensing element to sense a measure related to a cardiac strain in a heart resulting from contraction and relaxation of myocardium during a cardiac cycle. Based on the sensed strain, an output may be provided for use by a user of the system to select a segment of the heart for lead placement.

### CROSS REFERENCE TO RELATED APPLICATION

This application claims the benefit of U.S. Provisional Application No. 61/736,985, filed on Dec. 13, 2012, and U.S. Provisional Application No. 61/678,693, filed on Aug. 2, 2012, under 35 U.S.C. §119(e), the entire disclosures of which are herein incorporated by reference.

### TECHNICAL FIELD

This disclosure relates to medical devices and, more particularly, to cardiac lead implantation systems, devices, and methods for aiding in lead implantation.

### BACKGROUND

An implantable pacemaker may treat cardiac arrhythmias, heart failure, and/or other cardiovascular disorders by delivering electrical energy to the heart through one or more implantable leads. During the implantation procedure, an external (non-implantable) pacing and measuring device, sometimes referred to as a Pacing System Analyzer (PSA), is often connected to the implantable leads, and used to evaluate various pacing modes and/or parameters to help determine whether the leads are properly placed and to determine suitable pacing parameters. Once lead placement is complete, a pacemaker is typically connected to the implantable leads and subcutaneously implanted in the chest area. With the implantation procedure complete, an external programmer is often used to program the pacemaker via telemetry, using the set of suitable pacing parameters determined with the PSA or other pacing parameters, as desired.

### SUMMARY

This disclosure relates to medical devices and, more particularly, to cardiac lead implantation systems, devices, and methods for aiding in lead implantation. This disclosure describes techniques for assessing a location for left ventricle (LV) and/or right ventricle (RV) lead implantation. Using various techniques of this disclosure, an intravascular probe may be used to help determine the timing and/or magnitude of cardiac strain at a given location of the heart in order to assess a potential pacing site for LV and/or RV lead implantation prior to placement of an LV or RV pacing lead in the heart.

An illustrative system may include, for example, a probe having a proximal region and a distal region, with a sensor positioned along the distal region of the probe. The sensor may be any suitable sensor for detecting cardiac strain at or near the sensor, such as an ultrasound transducer, an optical sensor, a pressure sensor, an accelerometer, and/or a gyroscope. An external analyzer may be operatively coupled to the sensor of the probe. The external analyzer, which may be situated outside the body, may include an I/O port for receiving one or more signals from the sensor. The external analyzer may further include a strain analyzer that receives the one or more signals from the sensor and determines a measure related to the cardiac strain across at least one segment of the heart that results from contraction and relaxation of myocardium during at least one cardiac cycle. The external analyzer may output an indicator for use by a user of the system to aid in selection of a segment of the heart for lead placement. The measure related to the cardiac strain may include a measure related to the timing of the cardiac strain relative to the cardiac cycle of the heart, a measure related to the amplitude of the cardiac strain relative to the cardiac cycle of the heart, and/or any other suitable measure, as desired.

The system may also include at least one pacing electrode configured to deliver pacing pulses, and at least one sensing electrode configured to receive an electrical signal in response to the delivered pacing pulses. In some cases, the external analyzer may be configured to control delivery, via at least one pacing electrode, of at least one pacing pulse to the selected segment of the heart, and then determine, based on electrical signals received via the at least one sensing electrode in response to the at least one pacing pulse, whether the selected segment for pacing is an electrically viable pacing site.

This Summary is an overview of some of the teachings of the present application and not intended to be an exclusive or exhaustive treatment of the present subject matter. Further details about the present subject matter are found in the detailed description and appended claims. The scope of the present invention is defined by the appended claims and their equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 is an illustration of an example of a pacing system analyzer for use during implantation of cardiac pacing leads and portions of an environment in which the system is used;

FIG. 2 is a diagram illustrating an example intravascular probe and pacing system analyzer for use in pacing-site selection;

FIG. 3 illustrates an example probe with pacing electrodes;

FIG. 4 illustrates an example probe having a sensor positioned along a distal region;

FIG. 5 is a schematic cross-sectional side view showing an illustrative left ventricle of a heart;

FIG. 6 is a graph illustrating a measure of strain during a cardiac cycle for each of the six segments of the left ventricle of FIG. 5;

FIG. 7A shows an example six segment contribution array for a patient;

FIG. 7B shows the example six segment contribution array of FIG. 7A after lead placement and pacing;

FIG. 8A illustrates another example probe in a guidewire format;

FIG. 8B illustrates an example optical sensor for use with a probe;

FIG. 8C illustrates another example optical sensor for use with a probe;

FIG. 8D illustrates an example MEMS pressure sensor for use with a probe;

FIG. 9 illustrates an example method for evaluating the viability of a site for pacing;

FIG. 10A illustrates an example method for selecting a pacing site; and

FIG. 10B illustrates an example method for evaluating the viability of a site for pacing.

### DESCRIPTION

Non-responder rates for cardiac resynchronization therapy (CRT) implantations may be around 30%. Left ventricle (LV) and/or right ventricle (RV) pacing lead placement targeting could improve responder rates. The optimal pacing site, however, varies across patients. Hence, lead placement strategies exist to aid clinicians in their selection of an LV and/or RV pacing site. Some example current preoperative lead placement strategies include echocardiographic techniques, e.g., tissue Doppler imaging (TDI) or strain rate imaging (SRI), or magnetic resonance imaging (MRI). Current lead placement strategies during implantation can include, for example, perioperative hemodynamic optimization or perioperative electrical dyssynchrony minimization and capture threshold verification.

This disclosure relates to cardiac lead implantation systems, devices, and methods for aiding in lead implantation. More specifically, this disclosure relates to, among other things, techniques for assessing a location for implantation of a cardiac lead, such as a left ventricular (LV) lead or a right ventricular (RV) lead. In some instances, an intravascular probe can be used to help determine the timing and/or magnitude of cardiac strain at a given location of a heart in order to assess a potential pacing site for LV and/or RV lead implantation. In some cases, the intravascular probe can be used to help determine the site of latest peak strain as a candidate for LV and/or RV pacing. Predicting a favorable location for the LV and/or RV lead can, for example, help improve the efficacy of cardiac resynchronization therapy (CRT), but this is just one example.

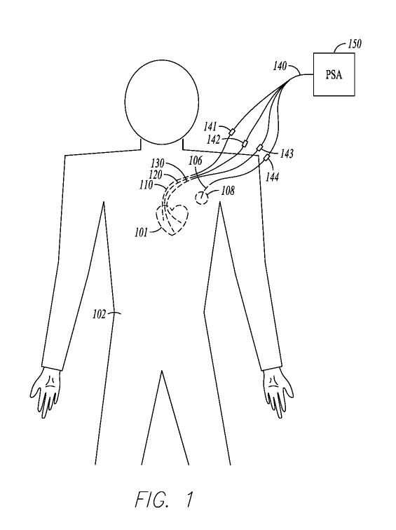

FIG. 1 is an illustration of an example of a pacing system analyzer for use during implantation of cardiac pacing leads, and portions of an environment in which the system is used. Before implantation of an implantable pacemaker into a body 102, portions of pacing leads 110, 120, and 130 are inserted into a heart 101. These pacing leads each include one or more electrodes for placement in or on heart 101 for sensing electrograms and delivering pacing pulses. Although the patient's body 102 is depicted as a human body, the techniques described in this disclosure are not limited to humans.

In the illustrated example, lead 106 is shown connected to a reference electrode 108, which can also be used for sensing electrograms and delivering pacing pulses. The electrodes of leads 110, 120, and 130 and electrode 108 are electrically connected to PSA 150 using a cable 140 with connectors 141, 142, 143, and 144. Connectors 141, 142, 143, and 144 are each configured for forming a temporary connection between cable 140 of the PSA 150 and the leads 110, 120, 130, and 106.

PSA 150 may be configured to perform tests that can include providing pacing pulses and taking measurements to help ensure that leads 110, 120, and 130 are properly positioned and, in some cases, to find a set of suitable pacing parameters. When the tests are completed, cable 140 may be disconnected from the leads, and lead 106 (with reference electrode 108) is removed from body 102. In the one example, an implantable CRT device may then be connected to leads 110, 120, and 130, and may be implanted into body 102 in approximately the location where electrode 108 was placed. The set of pacing parameters determined during the test may be programmed into the implantable pacemaker as the initial pacing parameters with which a pacing therapy begins. In some cases, the implantable pacemaker may have a housing that includes a conductive portion used as a reference electrode, replacing electrode 108.

The PSA 150 may include one or more individually controllable sensing and pacing channels. For example, the PSA 150 may include three individually controllable sensing and pacing channels. In one example, PSA 150 may include three individual sensing channels for sensing of atrial, RV, and/or LV electrograms, and three individually controllable pacing channels for delivery of atrial, RV, and/or LV pacing pulses. In a CRT application, PSA 150 may allow for pacing system testing before implantation of a CRT device having atrial, RV, and/or LV channels. In some cases, PSA 150 may perform various measurements and execute a CRT pacing algorithm using programmable pacing parameters including, but not limited to, atrioventricular and interventricular pacing delays. In some cases, PSA 150 may include four (or more) individually controllable sensing and pacing channels. A four-channel PSA 150 may, for example, test before implantation, a cardiac resynchronization therapy (CRT) device having atrial, RV, and two LV channels.

In some cases, and prior to insertion of an LV and/or RV lead 130 into the body 102, an intravascular probe may be used to help determine the timing and/or magnitude of cardiac strain at a given location of the heart in order to assess a potential pacing site for LV and/or RV lead implantation. In some cases, the intravascular probe can be used to help determine the site of latest peak strain as a candidate for LV and/or RV pacing. Once a desirable pacing location has been identified, an LV and/or RV lead can be delivered to the location and tested. Predicting a favorable location for the LV and/or RV lead can help improve the efficacy of cardiac resynchronization therapy (CRT), but this is just one example.

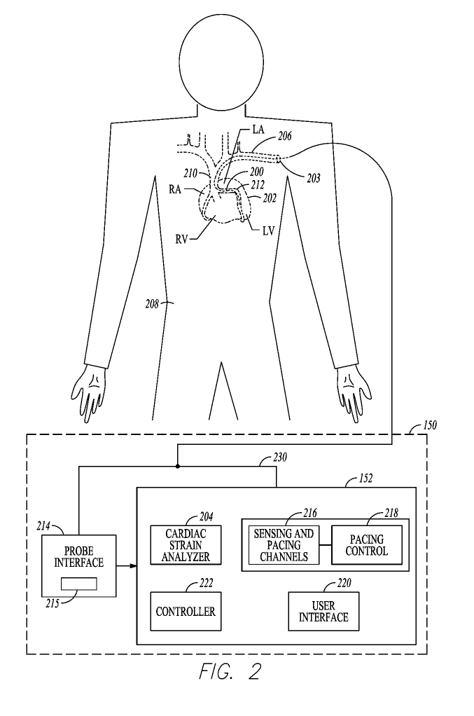

FIG. 2 is a diagram illustrating an example intravascular probe 200 and pacing system analyzer 150 for use in pacing-site selection. In accordance with various techniques described in this disclosure, the intravascular probe 200 may be advanced to a segment of the heart 202, and can be used to assess, for example, a segment for LV, RV and/or atrial lead implantation. In some cases, the pacing system analyzer 150 may include an analyzer module 152 and a probe interface 214. The probe interface 214 may communicate with a sensor that is positioned on a distal end of the intravascular probe 200. The pacing system analyzer 150 may include a cardiac strain analyzer 204, which may receive one or more signals from the sensor on the distal end of the intravascular probe 200, and may determine a measure related to cardiac strain across the at least one segment of the heart 202. The strain may result from contraction and relaxation of myocardium during a cardiac cycle.

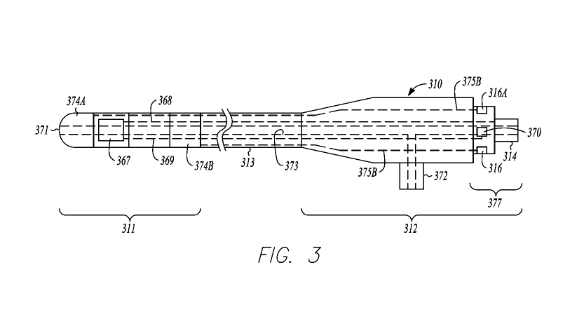

In the example configuration, the intravascular probe 200 may be an intravascular ultrasound (IVUS) mapping probe, and the sensor on the distal end of the intravascular probe 200 may include an IVUS transducer (see FIG. 3A). In one example, an IVUS mapping probe 200 may be delivered over a guidewire (not depicted) or by using a guidewire with ultrasound capability during a CRT LV lead placement procedure. Other procedures can be used to deliver the IVUS mapping probe 200 to a left ventricular coronary vein, as desired. During the procedure, an opening 203 may be made in a subclavian (or cephalic) vein 206 in a patient's body 208. An introducer (not depicted), e.g., a guide catheter, can be inserted into the subclavian vein 206, advanced to the superior vena cava 210, into the right atrium (RA) and then into the coronary sinus. Branches off of the coronary sinus reside on the epicardial surface of the left ventricle and can be areas of interest. Then, a guidewire (not depicted) can be inserted into the catheter and advanced into a coronary vein. If the guidewire does not have ultrasound capability itself, the IVUS mapping probe 200 can then be inserted over the guidewire and advanced to an area of interest of the heart 202.

In this example, an ultrasonic transducer at a distal region of the IVUS mapping probe 200 (or guidewire with ultrasound capability) can be used to visualize the segment of interest (e.g. left ventricle (LV) 212). In one example, a distal end portion of the IVUS mapping probe 200 (or guidewire with ultrasound capability) can include one or more pacing electrodes to allow pacing pulses to be delivered to a heart 202, as described in more detail below, but this is not required.

In some cases, the probe interface 214 may include an ultrasound interface, including a controller 215, which may generate ultrasound images using signals acquired by the IVUS mapping probe 200. An example probe interface 214 may include the iLab® Ultrasound Imaging System available from Boston Scientific Corporation (Natick, Mass. 01760). In some instances, the IVUS mapping probe 200 may be a pacing catheter formed by incorporating one or more pacing electrodes onto an IVUS catheter such as the Atlantis® SR Pro Coronary Imaging Catheter available from Boston Scientific Corporation, or a modified version thereof. In some cases, it may be desirable to modify the Atlantis® SR Pro Coronary Imaging Catheter to operate at a lower frequency (e.g. <12 MHz) to help visualize tissue that is further from the catheter, such as various walls of the heart and/or tissue that might move in response to undesirable nerve stimulation. In some instances, the Atlantis® SR Pro Coronary Imaging Catheter, or similar device, may be modified to operate at a variable frequency, including sufficiently low frequencies (e.g. <12 MHz), that is controllable by a user and/or the PSA 150.

Probe interface 214 and, more particularly, controller 215 may be in communication with the analyzer module 152 via communication link 230, which may be a wired or wireless link. The controller 215 can, for example, transmit ultrasound images generated by the probe interface 214 to analyzer module 152 and, in particular, the cardiac strain analyzer 204. In some examples, the probe interface 214 and the analyzer module 152 may be a single device or separate devices, as desired.

As indicated above and in accordance with various techniques of this disclosure, the analyzer module 152 may include the cardiac strain analyzer 204. The cardiac strain analyzer 204 can be configured to determine a location for left ventricular pacing based on cardiac strain and, in some examples, myocardial wall motion. Cardiac strain, or deformation, may refer to cardiac muscle contraction and relaxation during a cardiac cycle. In some cases, such as when the probe 200 is an IVUS mapping probe 200, analysis of cardiac strain can differentiate between active and passive mechanical motion. For example, healthy myocardium shortens during systole resulting in wall thickening. A scarred or ischemic region, in contrast, will remain thin as the tissue is stretched due to increased intracavitary pressure. Thus, wall thickening during systole can identify healthy tissue that positively contributes to the ejection of blood. A change in wall thickness can be detected using an IVUS mapping probe 200.

The amplitude of cardiac strain, e.g., using radial, circumferential and/or longitudinal strain, can be a strong predictor of acceptable LV lead placement because pre-exciting high strain regions can help normalize the strain around the ventricle. In a healthy heart, when assessed from a cross-sectional view, all the segments of the ventricle achieve approximately equal strain. In a failing heart, contraction often is distorted because the regions do not contract synchronously. The most delayed region will often attain its peak strain latest and this strain will have the greatest amplitude. As such, it can be desirable to place an LV pacing lead on the slowest segment of the left ventricle to pre-excite that segment so that all segments contract as synchronously and with as similar strain as possible. Lead placement informed by time to peak radial, circumferential and/or longitudinal strain, and/or by strain amplitude, can increase the likelihood and degree of a positive CRT response.

In some cases, the probe 200 may include a pressure sensor on a distal region of the probe. Sensed pressure may be related to cardiac strain. When so provided, the probe interface 214 may provide an interface between the pressure sensor and the analyzer module 152. In some cases, the pressure sensor may be an optical pressure sensor (e.g. Fiber Bragg Gratings, Fabry-perot), a mechanical pressure sensor with a sensing diaphragm (e.g. Micro-Electro-Mechanical System (MEMS) pressure sensor with a sensing diaphragm), a Surface Acoustical Wave (SAW) based pressure sensor, or any other suitable pressure sensor as desired. In some cases, the probe 200 may include an accelerometer and/or a gyroscope, which may detect motion. The detected motion may be related to cardiac strain by the cardiac strain analyzer 204. Also, the cardiac strain and/or motion may be used to detect undesirable nerve stimulation.

The example shown in FIG. 2 includes sensing and pacing channels 216, and pacing control 218. Sensing and pacing channels 216 can include, for example, three or more individually controllable sensing and pacing circuits for sensing from and delivering pacing pulses to three or more cardiac sites. Pacing control 218 can control the operation of sensing and pacing channels 216, including the delivery of the pacing pulses in each sensing and pacing channel.

The analyzer module 152 may include a user interface 220, which may be electrically connected to the electronic circuitry enclosed in the analyzer module 152. The user interface 220 may, for example, allow a user such as a physician or other caregiver to operate PSA 150 and observe information acquired by PSA 150.

The analyzer module 152 may further include a controller 222. The controller 222 can control the overall operation of the PSA 150 in accordance with programmed instructions and/or circuit configurations. The controller 222 (and the controller 215 of the probe interface 214) can be implemented as a microprocessor-based controller and can include a microprocessor and memory for data and program storage, implemented with dedicated hardware components such as ASICs (e.g., finite state machines), or implemented as a combination thereof. In one example, cardiac strain analyzer 204 may be stored or encoded as instructions in memory (not depicted) that are executed by the controller 222, but this is just one example.

The controller 222 can include timing circuitry such as clocks for implementing timers used to measure lapsed intervals and schedule events. As the term is used herein, the programming of the controller 222 refers to either code executed by a microprocessor or to specific configurations of hardware components for performing particular functions. Interfaced to the controller 222 are the sensing and pacing channels 216 and the pacing control 218 by which the controller 222 interprets sensing signals and controls the delivery of pacing pulses in accordance with a pacing mode.

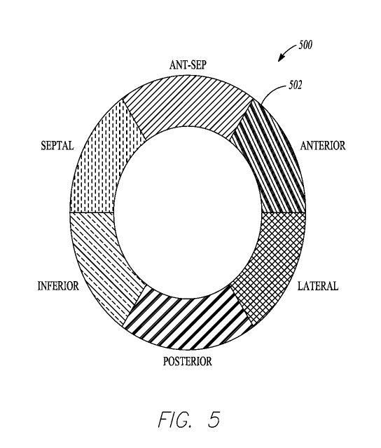

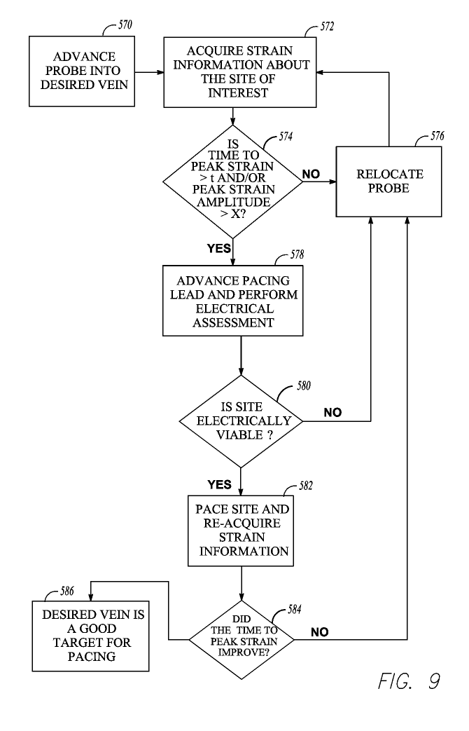

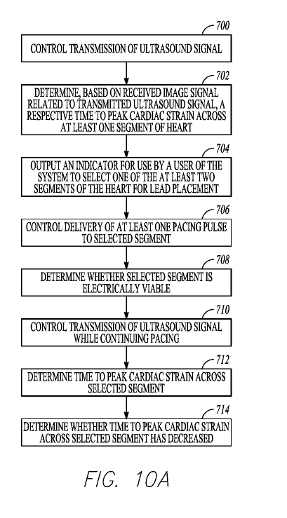

As described in detail below with respect to FIG. 9B, an IVUS-enabled speckle-tracking probe, e.g., probe 310 of FIG. 3, can be advanced over a guidewire, for example, through a vein of interest for mechanical assessment of a site for LV pacing. Probe interface 214 can control acquisition of data from a one-dimensional (similar to M-mode) or two-dimensional (B-mode) view of the left ventricle, for example, for speckle-tracking strain imaging. The controller 222 of analyzer module 152 can control the cardiac strain analyzer 204 to determine, for example, the timing for changes in strain for either or all of radial, circumferential and/or longitudinal LV strain. In one example, the cardiac strain analyzer 204 can separate the imaged left ventricle into a plurality of segments (see FIG. 5). Then, using electrical cardiac cycle information obtained by an electrocardiogram (ECG), the cardiac strain analyzer 204 can select a time in the cardiac cycle as an initial time and analyze how the speckles in one or more segments move relative to each other and to the cardiac cycle. Based on the relative motion, the cardiac strain analyzer 204 can determine the deformation of the various segment(s), and thus the relative amplitude or timing of the deformation in each segment. Also, and in some cases, the cardiac strain analyzer 204 may be configured to detect undesirable nerve stimulation.

The cardiac strain analyzer 204 can output an indicator for use by a user of the system, e.g., a clinician, to select one of the segments of the heart for the lead placement. For example, the cardiac strain analyzer 204 may output a visual indicator on a display of the user interface 220 of a segment of the heart for lead placement based on the determined relative amplitude of the segments. In another example, the cardiac strain analyzer 204 may select, without requiring user intervention, a segment of the heart for lead placement based on the relative timing of the cardiac strain of the two or more segments. In another example, the cardiac strain analyzer 204 may determine the amplitude or timing of the deformation in one segment and compare that amplitude or timing to a population model value retrieved from a memory device (not depicted). In one example, as long as the time to peak strain, for example, is greater than the population model retrieved, then the segment may be selected for lead placement.

If the site is determined to be desirable for lead placement based on the determined strain information, controller 222 may control delivery of pacing pulses to the site and receive sensing information in response to the delivered pacing pulses via the pacing control 218 and the sensing and pacing channels 216. In this manner, the electrical viability of the site may be assessed prior to LV lead implantation. If the site is electrically viable, strain amplitude and timing may be reassessed with pacing to determine whether the desired normalization is evident. At this time, the controller 222 can run through a series of different atrioventricular and intraventricular delays while monitoring strain amplitude and timing to determine how to program the device after implantation to achieve better or optimum normalization of strain timing and amplitude over the left ventricle. If the probe 200 is not the guidewire, the mapping probe can be removed and the LV lead can be inserted over the guidewire and implanted at the desired site.

In some cases, utilizing an IVUS-enabled catheter or guidewire for imaging cardiac strain can improve image quality over existing transthoracic or trans esophageal echocardiography techniques because the ultrasound beam is not scattered by intervening ribs and lungs as in transthoracic echocardiography.

FIG. 3 illustrates an example probe with pacing electrodes. While pacing electrodes are shown on the probe in FIG. 3, they are not required in all embodiments. In FIG. 3, the probe may be an IVUS-enabled speckle-tracking echocardiography catheter 310, which can be used to implement various techniques of this disclosure. Speckle-tracking echocardiography (also referred to herein as “speckle-tracking”), is an imaging technique that can analyze myocardial motion using ultrasound to track acoustic reflections and interference patterns (referred to collectively in this disclosure as “speckles”). These reflections and interference patterns can be assembled into strain-based waveforms that can be used to determine the relative timing and amplitude of radial, circumferential and/or longitudinal strain across a ventricle. The strain timing and amplitude information can allow assessment of the mechanical viability and resynchronization potential of a location prior to LV lead implantation. Also, the detected strain (and/or motion) may be used to detect undesirable nerve stimulation.

As described in more detail below, the IVUS-enabled speckle-tracking catheter 310 can include pacing electrodes that can deliver pacing pulses to assess the viability of a candidate site prior to implantation of an LV lead. The example IVUS catheter 310 includes a proximal region 312, a distal region 311 configured for intravascular placement, and an elongated shaft 313 coupled between proximal region 312 and distal region 311. In various example configurations, one or more pacing electrodes may be incorporated onto distal region 311 and/or shaft 313.

Proximal region 312 may include a catheter connector 377 and an injection port 372, but this is not required in all cases. Catheter connector 371 can include one or more connectors configured to provide mechanical and electrical connections between IVUS catheter 310 and PSA 150 of FIG. 2 (or an external pacemaker), a pullback motor (not depicted), and/or any other suitable device. In the example shown, catheter connector 377 includes a transducer connector 314 that provides electrical connection between the IVUS catheter 310 and the PSA 150. The connector 314 may further include pacing connectors (contacts) 316A-B that provide electrical connection between the pacing contacts and an external pacemaker.

Distal region 311 of the IVUS catheter 310 may include an ultrasonic transducer 367, pacing/sensing electrodes 374A-B, and an exit port 371. Ultrasonic transducer(s) 367 can transmit an ultrasound signal into the heart, and can receive an image signal related to the transmitted ultrasound signal. Pacing/sensing electrodes 374A-B, when provided, may be used to deliver pacing pulses and sense a physiologic response to the delivered pacing pulse. For illustrative purposes, two pacing electrodes are shown in FIG. 3. In various examples, distal region 311 can include one, two, or more pacing electrodes. In one example, distal region 311 can include at least one electrode for a unipolar pacing configuration with another electrode. In another example, distal region 311 of IVUS catheter 310 may include at least two electrodes for a bipolar pacing configuration. In one example, one or more additional pacing electrodes can be incorporated onto shaft 313. Thus, by way of example, the electrodes on the IVUS catheter 310 may simulate the electrodes on a cardiac lead to be implanted, such as quad-polar or other multipolar implantable cardiac leads. In one example, one or more of the pacing electrodes are configured to allow delivery of cardioversion/defibrillation pulses. Exit port 371, when provided, may allow liquid from the IVUS catheter 310 to exit the distal end.

Mechanical and electrical links can extend in shaft 313. In some cases, a rotating drive shaft 368 may be connected between ultrasonic transducer 367 and transducer connector 314 to allow ultrasonic transducer 367 to be driven by a pullback motor. An ultrasound lead 369 may be connected between ultrasonic transducer 367 and ultrasound connector 370, and can include multiple conductors to transmit signals to and from ultrasonic transducer 367. Conductors 375A-B can be connected between pacing/sensing electrodes 374A-B and pacing connectors 316A-B to conduct the pacing pulses. A lumen 373 can connect injection port 372 and exit port 371 to allow a liquid to flow through IVUS catheter 310, if desired. In one example, lumen 373 can accommodate a portion of a guide wire used to guide the insertion of IVUS catheter 310 to a desired site. Rotating drive shaft 368, ultrasound lead 369, conductors 375A-B, and lumen 372 are shown in FIG. 3 to illustrate various illustrative connections between components without necessarily reflecting their physical appearance and relative positions. It is contemplated that the IVUS catheter 310 may be made as a disposable device. One suitable IVUS catheter 310 may the Atlantis® SR Pro Coronary Imaging Catheter available from Boston Scientific Corporation, or a modified version thereof. In some cases, it may be desirable to modify the Atlantis® SR Pro Coronary Imaging Catheter to operate at a lower frequency (e.g. <12 MHz) to help visualize tissue that is further from the catheter, such as various walls of the heart and/or tissue that might move in response to undesirable nerve stimulation. In some instances, the Atlantis® SR Pro Coronary Imaging Catheter, or similar device, may be modified to operate at a variable frequency, including sufficiently low frequencies (e.g. <12 MHz), that is controllable by a user and/or the PSA 150.

In some cases, rather than using a rotating drive shaft 368 to rotate the ultrasonic transducer 367, one, two, or an array of non-rotating ultrasonic transducers or crystals can be arranged on IVUS catheter 310. Instead of rotating the ultrasonic transducers, each ultrasonic transducer can be turned on sequentially to produce a corresponding ultrasonic image. In addition, use of a phased-array design can advantageously reduce the size of the IVUS catheter 310, if desired. For example, it may be desirable to include differently tuned ultrasonic transducers or crystals in order to provide imaging immediately adjacent to the mapping probe as well as to provide imaging further away from the mapping probe, e.g., at the septum. In one example, a phased array of transducers or crystals can be configured to include one or more ultrasound transducers tuned to a first frequency and one or more ultrasound transducers tuned to a second frequency, where the transducers tuned to the first frequency are alternately arranged with the transducers tuned to the second frequency.

If a single crystal configuration is used and the IVUS catheter 310 is advanced into a coronary vein, and if the ultrasound transducer is aimed at the opposite wall, the ultrasound can pass through the wall with which it is in contact. Thus, the first reflected signal can give information about the near wall, and the later reflected signal can give information about the opposite wall of the ventricle. The two signals can be assesses simultaneously (if the time between reflections is long enough), or else the signals can be measured serially but fast enough to monitor wall thickness and wall motion on both walls over a complete cardiac cycle.

The pacing and sensing electrodes of the IVUS catheter 310 may be substantially similar to the pacing and sensing electrodes on an LV lead for implantation. For example, the pacing and sensing electrodes of the IVUS catheter 310 can be similar in surface area, lead spacing, polarization coating, number of poles, etc. to that of the pacing and sensing electrodes on an LV lead to be implanted. In this manner, the electrical measurements (e.g., capture threshold, R-wave amplitude, phrenic nerve stimulation threshold) made by the pacing and sensing electrodes of the IVUS catheter 310 can closely approximate the electrical performance of the LV lead to be implanted.

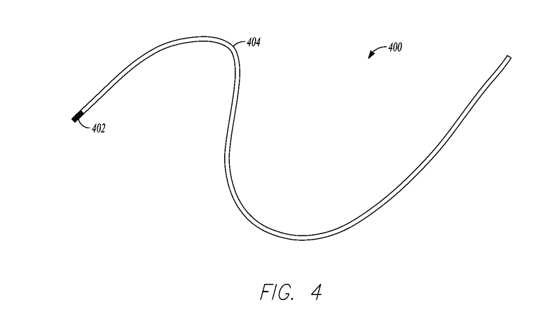

FIG. 4 illustrates another example of a probe. The probe 400 of FIG. 4 is an M-mode ultrasound device that utilizes a single crystal 402 or ultrasound transducer. Instead of using a rotating ultrasound transducer or phased-array of transducers to produce an image of one or more segments of the ventricle, as described above, the probe 400 may use the single crystal or ultrasonic transducer 402 to measure myocardial wall thickness at the location of the probe as a function of time. Hence, the probe 400 can have a reduced sized in comparison to the IVUS catheter 310 of FIG. 3, which use a rotating ultrasound transducer or phased-array of transducers.

In contrast to the example IVUS-enabled catheter shown and described above with respect to FIG. 3, the probe 400 of FIG. 4 may use myocardial wall motion in addition to cardiac strain to mechanically assess the viability of a pacing site. Probe interface 214 of FIG. 2 can control acquisition of data for a portion of the myocardial wall of the left ventricle and the septum, for example. The controller 222 of analyzer module 152 can control the cardiac strain analyzer 204 to, for example, analyze motion of the myocardial wall relative to the motion of the septum of the heart. The catheter can be positioned in a coronary vein, as previously described, at a candidate pacing site. While observing the ultrasound image, a clinician can rotate the probe until the motion of the near wall, usually either the lateral or posterior wall, and the septal wall are visible in the M-mode display. If wall thickening from the near wall appears sufficiently delayed (using a threshold that can be defined) with respect to the septal wall, the near wall can be paced at several different atrio-ventricular delays to determine whether contractions in the two regions can be synchronized. In some cases, the probe 400 itself may include a pacing electrode for this purpose. In other cases, a lead with one or more pacing electrodes may be slid over (or through) the probe 400 to approximate lead implantation, but so as not to obscure the ultra-sound image. If it is determined that contractions in the two regions can be synchronized, this can be identified as an adequate pacing site. If not, the probe 400 may be re-positioned in a new candidate pacing site, and the procedure repeated until an adequate pacing site is found.

The probe interface 214 can probe 400 to image the motion of the septum and the motion of a portion of the myocardial wall of the left ventricle at the location of the probe 400. In one example, the cardiac strain analyzer 204 can determine the relative timing between the septum wall and the myocardial wall by measuring the time from a feature in the septal wall motion signal to the time of the corresponding feature in the near wall.

In some cases, electrical information can be used. Using electrical cardiac cycle information obtained by an ECG, for example, the cardiac strain analyzer 204 may select a time in the cardiac cycle as an initial time and determine a first time between an R-wave and the motion of a portion of the myocardial wall of the left ventricle at the location of the probe 400. Next, using the electrical cardiac cycle information obtained by an ECG, the cardiac strain analyzer 204 may determine a second time between an R-wave and the motion of the septum. Based on a difference between the determined first and second times, the cardiac strain analyzer 204 may determine whether the LV lead should be placed at the location of the probe 400.

In one example, the cardiac strain analyzer 204 may compare the difference between the first and second times (the time between when the myocardial wall moves and when the septum moves) to a value stored in memory (e.g., from a population model). If the difference between the first and second times is greater than the value stored in memory, then the cardiac strain analyzer 204 may select the location of the probe 400 as a site for LV lead placement and display the selection to the user on the user interface 220. This candidate site may be paced to determine whether the time delay can be corrected.

In another example, the probe 400 may be formed with a guidewire. As such, no additional equipment may be necessary to perform the techniques described below with respect to FIG. 9A. For example, the ultrasound crystal or transducer and associated wiring of the probe 400 may be combined with a guidewire 404, and the guidewire 404 may be advanced as described above to a candidate site. The probe interface 214 of FIG. 2 can acquire an image of the ventricle via the ultrasound crystal or transducer 402. Once the acquired image has been mechanically assessed via the cardiac strain analyzer 204, then the LV lead can be delivered over the guidewire 404. The LV lead can then be used to electrically assess the viability of the candidate site.

In some cases, the single ultrasound crystal 402 can be positioned on a catheter rather than on a guidewire, as described above with respect to FIG. 3. In some cases, the single ultrasound crystal 402 can be positioned on an LV lead, rather than on a guidewire or catheter. When so provided, the crystal 402 may remain implanted with the LV lead, and may be used chronically to provide images to a clinician, if desired.

Cardiac strain analyzer 204 may determine a measure related to a cardiac strain signal resulting from contraction and relaxation of myocardium during the cardiac cycle across at least one segment of a heart based on a received image signal derived from the ultrasound transducer. As indicated above, the measure can be related to the timing and/or amplitude of the cardiac strain signals across one or more segments of the heart. In some cases, the measure can be related to an area-change contribution from one or more segments of the heart. In other cases, the measure can be related to a sensed pressure, motion or other parameter measured in or around a candidate pacing site, as further described below.

FIG. 5 is a schematic cross-sectional side view showing an illustrative left ventricle 500 of a heart. The ventricle wall 502 appears as approximately a circle with an enclosed area, e.g., the area of the chamber. The area has been subdivided into a number of segments. In FIG. 5, the left ventricle 500 has been divided into six segments that represent six regions of the left ventricle, e.g., anterior, posterior, lateral, inferior, septal, and antero-septal segments. In some examples, the left ventricle 500 can be divided into more than six segments, or less than six segments. In a healthy heart, each of the segments achieves approximately equal strain and contract synchronously.

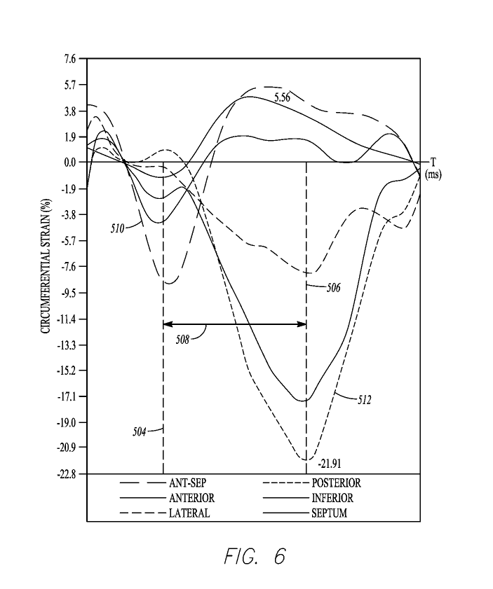

FIG. 6 is a graph illustrating a measure of strain during a cardiac cycle for each of the six segments of the left ventricle 500 of FIG. 5. In FIG. 6, the x-axis represents time (in milliseconds) and the y-axis represents a circumferential strain (in percent) for the anterior, posterior, lateral, inferior, septal, and antero-septal segments in FIG. 5. In a healthy heart, each of the signals shown in FIG. 6 would be in-phase and have similar amplitudes. As seen in FIG. 6, however, the signals are not all in-phase, indicating dyssynchrony between the segments of the left ventricle 500. In addition, there are large differences in amplitudes between the segments of the left ventricle 500. The dyssynchrony and amplitude differences depicted in FIG. 6 can be indicative of a heart that is failing.

Cardiac strain analyzer 204 can determine a measure related to the timing of the cardiac strain signals across one or more segments of the heart, e.g., left ventricle 500. For instance, cardiac strain analyzer 204 can determine a time to peak strain. FIG. 6 depicts two vertical lines, namely lines 504 and 506, which each correspond to a respective peak timing of two segments of the left ventricle. Cardiac strain analyzer 204 can determine the time between these peaks, e.g., the time between lines 504 and 506, as indicated by arrow 508. To improve the synchrony between the segments of the left ventricle 500, the time between peak strain, as indicated by arrow 508, should be reduced.

Rather than using a time to peak strain, cardiac strain analyzer 204 may determine a measure related to the timing of the cardiac strain signals across one or more segments of the heart, e.g., left ventricle 500, by using a covariance-based approach. As seen in FIG. 6, the signals have multiple peaks, and their relative amplitude can change from beat to beat. A covariance-based approach is one method to determine the relative timing information of the cardiac signals of the segments of left ventricle 500. More particularly, the covariance-based approach can be used to determine the phase shift between two signals or between a signal and some other standard, e.g., population model data.

In one example, the covariance between two signals, e.g., signals 510 and 512 of FIG. 6, can be computed, which is indicative of the phase angle, or phase shift, between the two signals. Then, one of the signals, e.g., signal 512, can be time-wise shifted and the covariance can be re-computed. When the covariance reaches a maximum value, the two signals are in phase. Using this covariance-based technique, the relative timing information for one or more cardiac strain signals can be determined and used to select a segment of the heart for lead placement for pacing.

In addition to timing, the cardiac strain analyzer 204 may use the magnitudes of the peaks of the cardiac strain signals to determine a measure related to a cardiac strain signal resulting from contraction and relaxation of myocardium during a cardiac cycle across at least one segment of a heart. Referring to FIG. 6, signal 512 has the largest peak magnitude, e.g., about −21.91, and hence the segment of the left ventricle corresponding to the signal 512 (posterior in this case) can be considered a candidate for lead placement.

Alternatively, or in addition to timing and amplitude techniques as described above, the cardiac strain analyzer 204 may use an area-change contribution technique to determine a measure related to cardiac strain resulting from contraction and relaxation of myocardium during a cardiac cycle across at least one segment of a heart. Such an area-change contribution technique may be based on a received image signal derived from a transmitted ultrasound signal. Referring again to FIG. 5, when progressing through a cardiac cycle, the overall enclosed area and the area of each of the segments changes, where the overall area change is equal to the sum of the segmental area changes. That is, as the ventricle wall 502 of the left ventricle 500 moves inward, not only is the overall enclosed area reduced, but the area in one or more of the six segments of the left ventricle 500 is also reduced.

It is desirable for each of the six regions of the heart to contribute equally to the overall area change of the left ventricle. In the example shown, it would be desirable for each of the six segments to contribute ⅙ or 0.167 of the overall area change. In a heart that is not healthy, and each of the six segments is represented as a different waveform, one waveform can indicate an increasing area while another waveform can indicate a decreasing area, thereby cancelling each other out, in effect. In general, late activated segments, e.g., the lateral or posterior wall in heart failure with left bundle branch block, may contribute more than a ⅙ share and early activated segments may contribute less than a ⅙ share. In some cases, pre-exciting a late activated segment with a pacing pulse can help reduce its contribution. It is desirable that late-activated segments activate earlier so that each of the six segments do the same or similar work.

In one example method of determining an area-change contribution, the cardiac strain analyzer 204 may determine a waveform that represents the overall area change, e.g., of the left ventricle 500. The cardiac strain analyzer 204 can determine the covariance between one or more waveforms that respectively represent the one or more of the segments against the overall area change. The segment with the largest covariance may make the greatest contribution to the overall area change, and is the prime candidate for lead placement.

One example technique for determining an area change contribution is described below. For each of the 6 exemplary segments shown in FIG. 5, the dot product between the area change in each segment from one ultrasound image frame to the next and the overall area change may be computed using Equation (1), below.

$${Contrib}_{{1}^{-}} := \frac{{\sum }_{j} {dATot}_{j} \cdot {dA}_{r , j}}{{\sum }_{j} {( {dATot}_{j} )}^{2}} {\sum }_{{1}^{-}} {Contrib}_{{1}^{-}} = 1 \qquad Equation ( 1 )$$

where dAr_{r,j} is the area change in region r from image frame j−1 to image frame j, dATotⱼ is the total area change from image frame j−1 to image frame j, and Contribᵣ is the fractional contribution to the overall area change from each of the r regions. The summation of all Contribᵣ equals 1, or the total area. In Equation (1), the numerator represents the covariance and the denominator represents the scaling factor of the total area change. Equation (1) generates a contribution array of the six segments, which includes the fractional contribution from each segment.

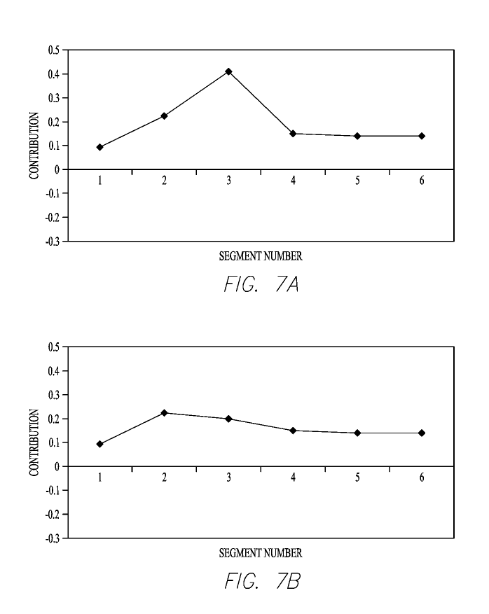

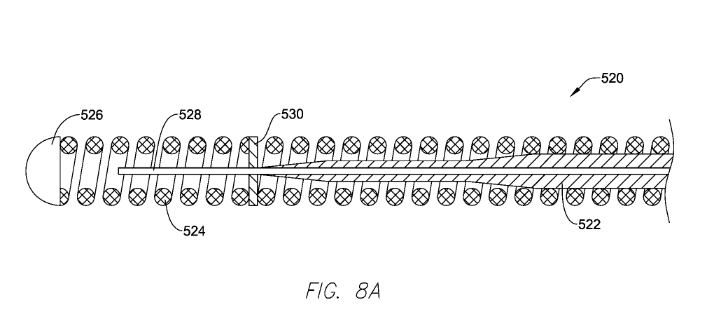

FIGS. 7A and 7B depict the six segment contribution arrays for a patient. In FIGS. 7A and 7B, the x-axis represents the segment number, and the y-axis represents the contribution of a segment. In FIG. 7A, segment 3 has the largest contribution of the six segments. Because segment 3 has the largest contribution, it may be the most desirable segment to select for lead placement for pacing. In one example implementation, the cardiac strain analyzer 204 may automatically select segment 3 for lead placement. In another example implementation, the cardiac strain analyzer may display the contributions of the segments, like in FIG. 6 or FIG. 7A, on a display and allow the clinician to manually select a segment for lead placement. FIG. 7B depicts the six segment contribution arrays after lead placement and pacing. As seen in FIG. 7B, the contribution of segment 3, which had the largest contribution in FIG. 7A before lead placement and pacing, has been reduced. In this manner, the contributions of the segments can be made more equal to one another, thereby helping to improve the performance of the heart.

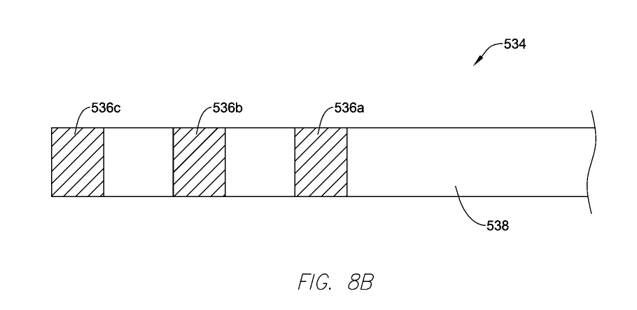

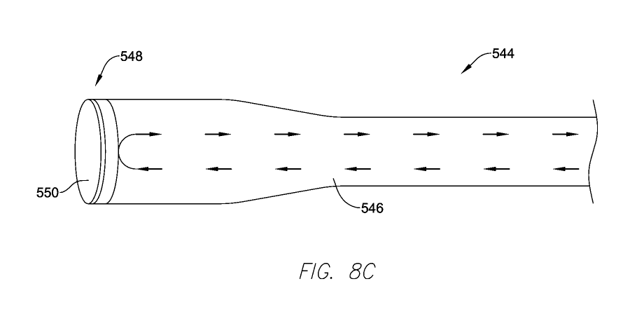

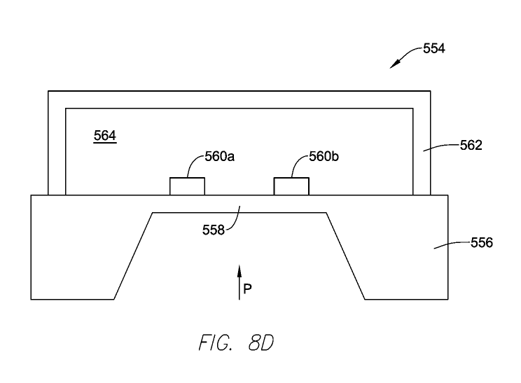

FIG. 8A illustrates another example probe. In FIG. 8A, the probe may include a pressure sensor for sensing a pressure at a candidate pacing site. The cardiac strain analyzer 204 (see FIG. 2) may convert the sensed pressure into a measure related to cardiac strain at the candidate pacing site. The measure of cardiac strain may be used to select an appropriate pacing site for lead placement. FIG. 8A shows a schematic cross-sectional view of an illustrative probe implemented as a guidewire 520. While implementation as a guidewire 520 is used as an example in FIG. 8A, it is contemplated that the probe may be implemented as a catheter (see FIG. 3), a pacing lead, or any other suitable device, as desired.

When implemented in a pacing lead, the sensing element may remain in the patient after a pacemaker is implanted, and may provide a mechanism for monitoring the resynchronization, decompensation and/or other parameters of the heart after the patient is released from the hospital. In such an instance, the pacemaker itself may be configured to include the functionality of the probe interface 204 and cardiac strain analyzer 214. In some cases, the pacemaker may also be configured to change one or more pacing parameters over time based on the sensed parameters.

Only a distal region of the guidewire 520 is shown in FIG. 8A. The illustrative guidewire 520 may include a core 522 that extends from a proximal end (not shown) of the guidewire 520 to near the distal end 526. A flexible coil 524 may be provided along the distal region of the guidewire 520 to help increase the flexibility in that region. A tip 526 is shown at the distal end of the guidewire 520. An optical fiber 528 is shown extending from the proximal end (not shown) of the guidewire 520, and down the core 522 toward the distal end. In the example shown, the optical fiber exits the core 522, and is exposed to the environment (e.g. cardiac pressure) in the distal region of the guidewire 520. In some cases, the distal end of the optical fiber may extend beyond the distal end of the guidewire. In some cases, the optical fiber may extend alongside the core 522, rather than through the core 522.

The probe interface 214 (see FIG. 2) may send input optical signals down the optical fiber, and may receive output optical signals in return. The cardiac strain analyzer 204 may determine a measure of sensed pressure along the distal region of the optical fiber by analyzing the output optical signals. The cardiac strain analyzer 204 may also convert a measure of sensed pressure into a measure related to cardiac strain along the distal region of the optical fiber (e.g. at a candidate pacing site). The measure of cardiac strain may then be used to select an appropriate pacing site for lead placement.

FIG. 8B illustrates an example optical sensor for use with a probe. The illustrative optical sensor 534 may include an optical fiber 538 that has one or more Fiber Bragg Gratings (FBG) 536a, 536b and 536c. Each FBG 536a, 536b and 536c may include a number of defined refractive index modulations (i.e. grating) along the length of the optical fiber. Each modulation may reflect a certain wavelength of an input optical signal provided by probe interface 214, and all the modulations may add coherently to produce an output wavelength (i.e. the Bragg wave) in an output optical signal. Pressure changes on the optical fiber may cause the spacing between the modulations of each FBG to change, which may change the output wavelength (Bragg wave) in the output optical signal. The cardiac strain analyzer 204 may determine a measure of sensed pressure along the distal region of the optical fiber by analyzing the wavelength of the output optical signals.

When more than one FBG is provided along the length of the optical fiber 538, each FBG 536a, 536b and 536c may be provided with a spacing between the defined refractive index modulations that is different from the other FBGs. The output optical signal may then include a different Bragg wave for each of the FBGs. This may allow the cardiac strain analyzer 204 to determine a measure of sensed pressure (and thus strain) at the locations of each of the FBG. This may help analyze several candidate pacing sites without repositioning the optical sensor 534, where each candidate pacing site corresponds to a corresponding FBG site along the optical fiber 538.

It is contemplated that the optical sensor shown in FIG. 8B may be incorporated into a guidewire, such as into the distal end of the optical fiber 528 of the guidewire 520, into a distal end of a catheter 320, into a distal end of a pacing lead such as a left ventricle pacing lead, and/or any other suitable device, as desired.

FIG. 8C illustrates another example optical sensor for use with a probe. The illustrative optical sensor 544 may include an optical fiber 546 with a Fabry-Perot (FP) cavity 548 positioned at or near a distal end of the optical fiber 546. The FP cavity 548 may include a diaphragm 550, which deflects under an applied pressure. When the diaphragm 550 deflects, the spacing of the FP cavity changes, which changes the resonance frequency of the FP cavity 548. A probe interface 214 may provide an input optical signal down the optical fiber 546 and to the FP cavity 548. The FP cavity 548 may reflect a wavelength and provide an output optical signal that is dependent on the resonance frequency of the FP cavity 548. The cardiac strain analyzer 204 may determine a measure of applied pressure to the FP cavity 548 by analyzing the wavelength of the output optical signals.

It is contemplated that the optical sensor shown in FIG. 8C may be incorporated into a guidewire, such as into the distal end of the optical fiber 528 of the guidewire 520, into a distal end of a catheter 320, into a distal end of a pacing lead such as a left ventricle pacing lead, and/or any other suitable device, as desired.

FIG. 8D illustrates an example MEMS pressure sensor 554 for use with a probe. The illustrative MEMS pressure sensor 554 includes a substrate 556 that defines a diaphragm 558. A cap 562 may form a sealed cavity 564 around a top side of the diaphragm 558, which applies a reference pressure to the top side of the diaphragm 558. A sensed pressure “P” may be applied to the bottom side of the diaphragm 558, which causes the diaphragm 558 to deflect when the sensed pressure “P” is different from the reference pressure in the cavity 564. In some cases, one or more piezoresistive elements 560a, 560b may be positioned on the diaphragm 558. The one or more piezoresistive elements 560a, 560b may be connected in a Wheatstone bridge configuration, such that the one or more piezoresistive elements can be used to sense the deflection of the diaphragm caused by the sensed pressure “P”. The MEMS pressure sensor 554 may be provided at or near the distal end of a probe. When so provided, the cardiac strain analyzer 204 may determine a measure of sensed pressure “P” by analyzing the output of the Wheatstone bridge. In some instances, more than one MEMS pressure sensors 554 may be positioned at, for example, spaced locations along the probe. When so provided, the cardiac strain analyzer 204 may determine a measure of sensed pressure “P” at the various spaced locations along the probe.

In another example, a pressure sensor may be a Surface Acoustical Wave (SAW) pressure sensor, which has a piezoelectric substrate patterned with one or more interdigitated electrodes. A sensed pressure may be applied to the piezoelectric substrate, which may change the speed or velocity of an acoustical wave traveling down the piezoelectric substrate. The patterned interdigitated electrodes may be used to induce an acoustical wave down the piezoelectric substrate, and to sense a received signal that can be analyzed to determine the speed or velocity of the induced acoustical wave in the piezoelectric substrate, which can be related to the sensed pressure. In some cases, the SAW pressure sensor may include one or more antennae, which can be used to both power and interrogate the SAW sensor wirelessly, sometimes via the probe interface 214 of FIG. 2.

In another example, a MEMS based accelerometer and/or gyroscope may be coupled to a distal region of a probe, and may detect motion at a candidate pacing site. The detected motion, particularly with respect to a cardiac cycle, may be related to cardiac strain by the cardiac strain analyzer 204.

It is contemplated that the MEMS pressure sensor 554, SAW pressure sensor, accelerometer and/or gyroscope may be incorporated into a guidewire, into a distal end of a catheter 320, into a distal end of a pacing lead such as a left ventricle pacing lead, and/or any other suitable device, as desired.

FIG. 9 illustrates another example of a method for evaluating the viability of a site for pacing. In the example flow diagram of FIG. 9, a clinician, e.g., physician, can advance a probe into a desired body lumen (570), e.g., vein. After the probe is positioned at the desired site (e.g. candidate pacing site), the analyzer module 152 of FIG. 2 may acquire strain information about the candidate site. It is contemplated that any of the probes discussed herein may be used to measure and/or acquire strain information about the candidate site, including using a probe that has one or more pressure sensors, an accelerometer, a gyroscope, an ultrasound transducer and/or any other suitable sensor, as desired.

In one example, the IVUS-enabled speckle-tracking catheter 310 of FIG. 3 can be advanced over a guidewire to the candidate site. In this example, and once the probe is positioned at the desired site, the probe interface 214 can be used to acquire an IVUS image of the LV (804) and/or it can image the wall motion, e.g., using a single crystal or transducer. In some cases, the controller 222 of the analyzer module 152 may be used to control the cardiac strain analyzer 204 to analyze motion of the myocardial wall relative to the motion of the septum. In addition to wall motion, the controller 222 of analyzer module 152 may control the cardiac strain analyzer 204 to determine a time to peak strain for radial, circumferential and/or longitudinal LV strain for one or more cardiac segments of the LV.

In some cases, the cardiac strain analyzer 204 may determine whether a cardiac segment has a time to peak strain greater than a threshold value “t” and/or the peak strain amplitude is greater than a threshold value “X”, such as shown in block 574. If the time to peak strain at the current location is not greater than a threshold value “t”” and/or the peak strain amplitude is not greater than a threshold value “X” (“NO” branch of block 574), then, if possible, the clinician can relocate the probe to another segment, searching for a time to peak strain greater than a threshold value “t”” and/or a peak strain amplitude greater than a threshold value “X”, as shown block 576. The threshold value “t” can be a threshold time to peak strain value that, if long enough, can indicate that the location is suitable for pacing. Likewise, the threshold value “X” can be a threshold strain amplitude value that, if sufficiently high, can also indicate that the location is suitable for pacing. These thresholds can be patient-specific values, or in some examples, can be values based on a model.

If the time to peak strain at the candidate location is greater than a threshold value “t” and/or the peak strain amplitude is greater than a threshold value “X” (“YES” branch of block 574), then in some cases, a pacing lead may be advanced to the current location to perform an electrical assessment of the candidate site, as shown at block 578. When the sensing probe is incorporated in a pacing lead (i.e. when a sensor is incorporated into a pacing lead), this step may not be necessary. Also, if the probe itself has one or more pacing electrodes, a lead may not be required to perform the electrical assessment. In any event, the controller 222 may control delivery of pacing pulses to the pacing electrode and may receive sensing information in response to the delivered pacing pulses via the pacing control 218 and the sensing and pacing channels 216 of analyzer module 152 (FIG. 2). If the controller 222 determines, based on the one or more determined parameters such as LV threshold, a presence of phrenic stimulation, an impedance, an intrinsic amplitude, and/or a Q-LV timing, that the candidate site is not electrically viable (“NO” branch of block 580), then the clinician may relocate the probe to another segment, as shown at block 576.

If the controller 222 determines, based on one or more of the determined parameters, that the site is electrically viable (“YES” branch of block 580), then the controller 222 may control delivery of pacing pulses to the site while also re-acquiring strain information, as shown at block 582.

If the time to peak strain and/or the peak strain amplitude of the segment has not sufficiently improved (“NO” branch of block 584), then the clinician may relocate the probe to another segment for assessment, as shown at block 576. If the time to peak strain of the segment and/or the peak strain amplitude has sufficiently improved (“YES” branch of block 584), then the site may be confirmed to be a mechanically and electrically viable location for LV lead placement, as shown at block 586. As mentioned above, the slowest segment will have hit its peak strain latest. As such, it can be desirable to place an LV pacing lead on the slowest segment so that pacing can hasten the slowest segment and cause all segments to contract as close in time to each other as possible. Strain-guided atrial-ventricular (AV) delay and ventricular-ventricular (VV) delay measurements can also be made, once the atrial and RV leads are implanted, in order to determine how to optimize either or both of the AV and VV delay to improve overall cardiac strain. In the manner, the mechanical dyssynchrony of a candidate site of an LV (and/or RV) can be assessed for delivery of pacing pulses by an LV (and/or RV) lead.

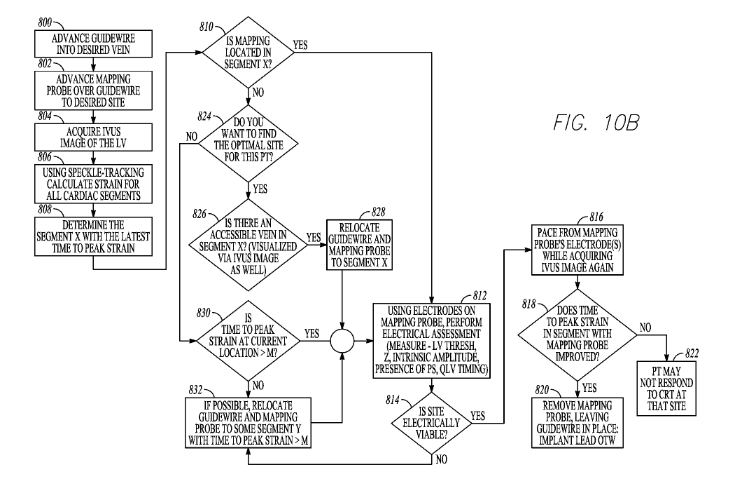

FIG. 10A illustrates an example method for selecting a pacing site. In the example method depicted in FIG. 10A, a controller 215 (FIG. 2) can control transmission of an ultrasound signal (700) from an ultrasound transducer in a probe. The cardiac strain analyzer 204 (FIG. 2) can determine, based on a received image signal related to the transmitted ultrasound signal, a respective time to peak cardiac strain across one or more segments of a heart (702), and output an indicator for use by a user of the system to select one of the at least two segments of the heart for the lead placement (704). For example, cardiac strain information for a single segment can be compared to cardiac strain information for a population model retrieved from a memory device. In another example, two or more segments of the patient's heart can be compared to one another either serially or substantially simultaneously. In this manner, the method of FIG. 10A depicts mechanically assessing the viability of a pacing site.

Optionally, the method of FIG. 10A can include additional steps. The controller 222 can control delivery, via the at least one pacing electrode, of at least one pacing pulse to the selected segment (706) and can determine, based on electrical signals received via the at least one sensing electrode in response to the at least one pacing pulse, whether the selected segment for pacing is electrically viable (708). The controller 215 (FIG. 2) can control transmission of another ultrasound signal (710) while pacing continues. The controller 222 of analyzer module 152 may determine, based on a received image signal related to the other transmitted ultrasound signal, a time to peak cardiac strain across the selected segment (712). Then, the controller 222 of analyzer module 152 may determine whether the initially determined time to peak cardiac strain across the selected segment has decreased in response to the delivery of the another pacing pulse to the selected segment (714). In other words, the controller 222 may confirm that the time to peak cardiac strain has improved. It may also assess timing at other sites to determine that pacing has resulted in overall improved synchrony.

FIG. 10B illustrates an example method for evaluating the viability of a site for pacing. In the example flow diagram of FIG. 10B, a clinician, e.g., physician, can advance a guidewire into a desired body lumen (800), e.g., vein. After the guidewire is in position, the clinician can advance a probe to a desired site. For example, the IVUS-enabled speckle-tracking catheter 310 of FIG. 3 can be advanced over a guidewire to the desired site.

Once the probe is positioned at the desired site, the probe interface 214 can acquire an IVUS image of the LV (504) or it can image the wall motion, e.g., using a single crystal or transducer. The controller 222 analyzer module 152 may control the cardiac strain analyzer 204 to use speckle-tracking in ultrasound images, for example, to determine a time to peak strain for radial, circumferential and/or longitudinal LV strain for a plurality of cardiac segments of the LV, e.g., six segments based on the acquired IVUS image. From the plurality of cardiac segments, the cardiac strain analyzer 204 may determine a cardiac segment with the latest time to peak strain. In the manner, the mechanical dyssynchrony of a site of an LV can be assessed for delivery of pacing pulses by an LV lead.

In one example implementation of the techniques of this disclosure, if the probe, e.g., probe 310 of FIG. 3, is located in a segment determined by the cardiac strain analyzer 204 to have the latest time to peak strain (“YES” branch of block 810), the clinician can remove the mapping probe, leave the guidewire in place, and implant the pacing lead over the guidewire (OTW). In this manner, the mechanical viability of the site can be assessed without assessing the electrical viability of the site. For purposes of clarity, this path is not depicted in the flow diagram of FIG. 10B. However, the newly deployed pacing lead can be used to provide electrical assessment of the site.

In another example, if the mapping probe, e.g., probe 310 of FIG. 3, is located in a segment determined by the cardiac strain analyzer 204 to have the latest time to peak strain (“YES” branch of block 810), then the controller 222 can control delivery of pacing pulses to the site and receive sensing information in response to the delivered pacing pulses via the pacing control 218 and the sensing and pacing channels 216 of analyzer module 152 (FIG. 2), as shown at block 812. Upon receiving the sensed information in response to the delivered pacing pulses, the controller 222 may assess the electrical viability of the site by determining, for example, one or more parameters such as an LV threshold, a presence of phrenic stimulation, an impedance, an intrinsic amplitude, and a Q-LV timing, and comparing one or more of the respective parameters to a respective value stored in a memory device.

If the controller 222 determines, based on one or more of the determined parameters, that the site is electrically viable (“YES” branch of block 814), then the controller 222 may control delivery of pacing pulses to the site while also controlling the probe interface 214 to acquire additional IVUS images of the site, as shown at block 816. The controller 222 may control the cardiac strain analyzer 204 to use speckle-tracking in the ultrasound images, for example, to determine, a time to peak strain for either or all of radial, circumferential and/or longitudinal LV strain for the site. In addition, the controller 222 may determine whether the time to peak strain for the segment has improved, as shown at block 818. As mentioned above, the slowest segment will have hit its peak strain latest. As such, it can be desirable to place an LV pacing lead on the slowest segment so that pacing can hasten the slowest segment and cause all segments to contract as close in time to each other as possible.

If the time to peak strain of the segment has improved (“YES” branch of block 818), then the site has been confirmed to be a mechanically and electrically viable location for LV lead placement. Strain-guided atrial-ventricular (AV) delay and ventricular-ventricular (VV) delay measurements can be made at this point, once the atrial and RV leads are implanted, in order to determine how to optimize either or both of the AV and VV delay to improve the cardiac strain.

The clinician can remove the probe, if present, leave the guidewire in place, and deliver an LV lead over the guidewire for implantation, as shown at block 820. If the time to peak strain of the segment has not improved (“NO” branch of block 818), then the patient may not respond to CRT treatment (822) at that location. The clinician can reposition the LV lead within the vein, try another branch of the vein, or try another vein.

If the controller 222 determines, based on the one or more determined parameters such as LV threshold, a presence of phrenic stimulation, an impedance, an intrinsic amplitude, and/or a Q-LV timing, that the site is not electrically viable (“NO” branch of block 814), then if possible, the clinician may relocate the guidewire and the probe to another segment with a time to peak strain greater than a threshold value “t” and/or the peak strain amplitude is greater than a threshold value “X”, as shown at block 832. The threshold value “t” can be a threshold time to peak strain value that, if long enough, can indicate that the location is suitable for pacing. Likewise, the threshold value “X” can be a threshold strain amplitude value that, if sufficiently high, can also indicate that the location is suitable for pacing. These thresholds can be patient-specific values, or in some examples, can be values based on a model.

In some examples, the image resolution can allow the entire ventricle to be imaged so that even if the probe 310 is not located in the vessel with the peak strain segment, the clinician can see where the peak strain segment is located and relocate the probe to that particular vessel, if desired. This can help reduce guesswork and save time during the procedure.

If no other mechanically delayed segment exists, the patient may not respond to CRT. If there is another segment with a time to peak strain greater than a determined value M and/or the peak strain amplitude is greater than a threshold value “X” (“YES” branch of block 832), once the probe is relocated, the controller 222 may control delivery of pacing pulses to the site and receive sensing information in response to the delivered pacing pulses via the pacing control 218 and the sensing and pacing channels 216 of analyzer module 152 (FIG. 2), as shown at block 812. The flow diagram continues at 814 as described above and, for purposes of conciseness, will not be described again.

Returning to near the top of the flow chart in FIG. 10B at 810, if the probe is not located in the segment determined by the cardiac strain analyzer 204 to have the latest time to peak strain (“NO” branch of block 810), then another site for LV placement can be determined, as shown at block 824. If the clinician wants to determine the other site for the patient (“YES” branch of block 824), then the clinician can determine, e.g., via an IVUS image, if there is an accessible vein in the segment previously assessed as having the latest time to peak strain (826) and then relocate the guidewire and probe to that segment (828). In some examples, the clinician can attempt to reach this other site by using information previously obtained by mapping the cardiac veins via dye injection during a fluoroscopy procedure. Then, the controller 222 may control delivery of pacing pulses to the site and receive sensing information in response to the delivered pacing pulses via the pacing control 218 and the sensing and pacing channels 216 of analyzer module 152 (FIG. 2), as shown at block 812. The flow diagram continues at 814 as described above and, for purposes of conciseness, will not be described again.

If the clinician does not want to determine the optimal site for the patient (“NO” branch of block 824), then the clinician can determine, e.g., via the cardiac strain analyzer 204 of FIG. 2, if the time to peak strain at the current location is greater than a threshold value “t” (830) and/or the peak strain amplitude is greater than a threshold value “M”. If the time to peak strain at the current location is not greater than a threshold value “M” and/or the peak strain amplitude is greater than a threshold value “A”, then, if possible, the clinician may relocate the guidewire and the probe to another segment “Y” with a time to peak strain greater than a threshold value “M” and/or with a peak strain amplitude greater than threshold value “A”, as shown at block 832. Again, the threshold value “M” can be a threshold time to peak strain value that, if long enough, can indicate that the location is suitable for pacing. Likewise, the threshold value “A” can be a threshold strain amplitude value that, if sufficiently high, can also indicate that the location is suitable for pacing. These thresholds can be patient-specific values, or in some examples, can be values based on a model.

If the time to peak strain at the current location is greater than a threshold value “M” and/or the peak strain amplitude is greater than threshold value “A” (“YES” branch of block 830), then the controller 222 may control delivery of pacing pulses to the site and receive sensing information in response to the delivered pacing pulses via the pacing control 218 and the sensing and pacing channels 216 of analyzer module 152 (FIG. 2), as shown at block 812.

In other words, prior to selecting, without requiring user intervention, a segment of the heart for LV lead placement based on the determined respective times to peak cardiac strain, the controller 222 may determine, via the cardiac strain analyzer 204, which of the respective times to peak cardiac strain is longest. The controller 222 can compare the longest time to peak cardiac strain to a threshold value, and if the longest time to peak cardiac strain is greater than the threshold value “M”, the controller 222 may control delivery, via the at least one pacing electrode, of at least one pacing pulse to the selected segment. Based on electrical signals received via the at least one sensing electrode in response to the at least one pacing pulse, the controller 222 may determine whether the selected segment for pacing is electrically viable. A similar approach may be applied to the peak strain amplitude, if desired. The flow diagram continues at 814 as described above and, for purposes of conciseness, will not be described again.

In this manner, the mechanical and electrical viability of a potential LV pacing site can be assessed prior to LV lead fixation. Once the viability of the site has been assessed and the site has been confirmed to be viable, the LV lead can be implanted and tested via the PSA 150. Then, the PSA 150 can be disconnected from the leads, and the CRT device can be implanted in the patient.

In some aspects, the perioperative ultrasound imaging techniques of the disclosure may enable accurate LV lead placement. In other aspects, the concurrent imaging capabilities described in this disclosure can mitigate the imperfections of existing imaging techniques and can reduce or even eliminate the need for preoperative imaging. In another aspect, the techniques of this disclosure may provide the clinician implanting the LV lead with fine, local, real-time, mechanical and electrical feedback. In yet another aspect, the techniques of this disclosure may be combined with an LV lead or LV lead delivery catheter or guidewire, thereby resulting in one less step for a clinician in a CRT implant process. In yet another aspect, the techniques of this disclosure can help differentiate CRT responders from patients that may not respond to CRT.

### Additional Notes and Examples

Modules and other circuitry shown and described herein may be implemented using software, hardware, firmware and/or combinations thereof. Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods.

The above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for aiding selection of a pacing site for lead placement in a heart, the system comprising:
a probe including:
a proximal region and a distal region;
a sensor positioned along the distal region of the probe;
an external analyzer operatively coupled to the sensor of the probe, the external analyzer including:
an I/O port for receiving one or more signals from the sensor;
a strain analyzer coupled to the I/O port, the strain analyzer receiving the one or more signals from the sensor via the I/O port and determining a measure related to a cardiac strain across at least one segment of the heart resulting from contraction and relaxation of myocardium during at least one cardiac cycle; and
an output coupled to the strain analyzer for outputting an indicator for use by a user of the system to select a segment of the heart for lead placement.

2. The system of claim 1, wherein the measure related to the cardiac strain includes a measure related to the timing of the cardiac strain relative to the at least one cardiac cycle of the heart.

3. The system of claim 1, wherein the measure related to the cardiac strain includes a measure related to the amplitude of the cardiac strain relative to the at least one cardiac cycle of the heart.

4. The system of claim 1, wherein the external analyzer provides one or more signals to the sensor via the I/O port.

5. The system of claim 1, wherein the sensor includes an ultrasound transducer.

6. The system of claim 1, wherein the sensor includes an optical sensor.

7. The system of claim 6, wherein the optical sensor includes a Fiber Bragg Grating (FBG).

8. The system of claim 7, wherein the Fiber Bragg Grating (FBG) is situated along an optical fiber, and the optical fiber extends along the probe and to the I/O port of the external analyzer, the external analyzer providing one or more optical signals to the FBG via the optical fiber, and receiving one or more return signals from the FBG, wherein the one or more return signals are dependent on an applied pressure to the FBG, which is related to cardiac strain.

9. The system of claim 1, wherein the sensor includes a pressure sensor.

10. The system of claim 9, wherein the pressure sensor includes a diaphragm with one or more piezoresistive elements in a circuit configuration, wherein the one or more piezoresistive elements sense a deflection of the diaphragm caused by an applied pressure to the diaphragm, which is related to cardiac strain.

11. The system of claim 9, wherein the pressure sensor includes a Fabry-Perot (FP) cavity that includes a diaphragm, wherein a resonant frequency of the Fabry-Perot cavity changes as the diaphragm deflects in response to an applied pressure to the diaphragm, which is related to cardiac strain.

12. The system of claim 11, wherein the Fabry-Perot (FP) cavity is optically coupled to an optical fiber, and the optical fiber extends along the probe and to the I/O port of the external analyzer, the external analyzer providing one or more optical signals to the FP cavity via the optical fiber, and receiving one or more return signals that are dependent on an applied pressure to the diaphragm, which is related to cardiac strain.

13. The system of claim 9, wherein the pressure sensor includes a Surface Acoustical Wave (SAW) pressure sensor having a piezoelectric substrate patterned with interdigitated electrodes.

14. The system of claim 1, wherein the probe includes a guide wire suitable for guiding a pacing lead to a pacing site in the heart.

15. A system for aiding selection of a pacing site for lead placement in a heart, the system comprising:
a probe including:
a proximal region and a distal region;
an ultrasound transducer positioned along the distal region of the probe, wherein the ultrasonic transducer is configured to transmit an ultrasound signal and receive an image signal related to the transmitted ultrasound signal;
an external analyzer coupleable to the ultrasound transducer of the probe, the external analyzer including:
an I/O port for receiving one or more signals from the ultrasound transducer;
a strain analyzer coupled to the I/O port, the strain analyzer receiving the one or more signals from the ultrasound transducer via the I/O port and determining a measure related to a cardiac strain across at least one segment of a heart resulting from contraction and relaxation of myocardium during at least one cardiac cycle; and
an output coupled to the strain analyzer for outputting an indicator for use by a user of the system to select a segment of the heart for lead placement.

16. The system of claim 15, further comprising:
at least one pacing electrode configured to deliver pacing pulses; and
at least one sensing electrode configured to receive an electrical signal in response to the delivered pacing pulses,
wherein the external analyzer is configured to:
control delivery, via the at least one pacing electrode, of at least one pacing pulse to the selected segment of the heart; and
determine, based on electrical signals received via the at least one sensing electrode in response to the at least one pacing pulse, whether the selected segment for pacing is an electrically viable pacing site.

17. The system of claim 16, wherein the external analyzer is further configured to:
control delivery, via the at least one pacing electrode, of another pacing pulse to the selected segment of the heart;
receive one or more signals from the ultrasound transducer;
determine, based on the received one or more signals, a time to peak cardiac strain across the selected segment; and
determine whether the determined time to peak cardiac strain across the selected segment has decreased in response to the delivery of the another pacing pulse to the selected segment.

18. The system of claim 15, wherein the strain analyzer is configured to determine the measure related to a cardiac strain across at least one segment of a heart resulting from contraction and relaxation of myocardium during a cardiac cycle by determining, using speckle-tracking, a relative motion between a plurality of speckles in the at least one segment of the heart during a cardiac cycle.

19. A system for aiding selection of a pacing site for lead placement in a heart, the system comprising:
a probe including:
a proximal region and a distal region;
an optical pressure sensor positioned along the distal region of the probe;
an external analyzer coupleable to the optical pressure sensor of the probe, the external analyzer including:
an I/O port for receiving one or more signals from the optical pressure sensor;
a strain analyzer coupled to the I/O port, the strain analyzer receiving the one or more signals from the optical pressure sensor via the I/O port and determining a measure related to a cardiac strain across at least one segment of a heart resulting from contraction and relaxation of myocardium during at least one cardiac cycle; and
an output coupled to the strain analyzer for outputting an indicator for use by a user of the system to select a segment of the heart for lead placement.

20. The system of claim 19, further comprising:
at least one pacing electrode configured to deliver pacing pulses; and
at least one sensing electrode configured to receive an electrical signal in response to the delivered pacing pulses,
wherein the external analyzer is configured to:
control delivery, via the at least one pacing electrode, of at least one pacing pulse to the selected segment of the heart; and
determine, based on electrical signals received via the at least one sensing electrode in response to the at least one pacing pulse, whether the selected segment for pacing is an electrically viable pacing site.
